(19) United States
(12) Patent Application Publication
(10) Pub. No.: US 2014/0366878 A1
(43) Pub. Date: Dec. 18, 2014

(54) MEDICAL CONSOLE

(71) Applicant: NetScientific Ltd, London (GB)

(72) Inventor: Ehud Baron, Tokyo (JP)

(21) Appl. No.: 14/360,381

(22) PCT Filed: Nov. 21, 2012
(86) PCT No.: PCT/GB2012/052879

§ 371 Date: May 23, 2014

(30) Foreign Application Priority Data
Nov. 25, 2011 (GB) 1120345.2

(51) Int. Cl. A61M 5/172; A61M 16/00

(52) U.S. Cl. CPC A61M 5/1723; A61M 16/0003; A61M 2205/3303; A61M 2205/50; A61M 2205/52; A61M 2230/04; A61M 2230/30
USPC 128/204.23; 604/66

(57) ABSTRACT

A personal medical console is connectable to a plurality of different sensor devices and different effector devices and configured to monitor operation of one or more sensor devices and control operation of one or more effector devices. The sensor devices sense physiological characteristics. The medical console is configured to receive sensor data from sensor devices and output control data defining one or more actions for controlling the one or more of the effector devices. An analysis engine in the medical console is configured to process the sensor data received from the one or more sensor devices and output control data in response to the sensor data by determining a divergence of the sensor data to a target health state, and generating the control data for the one or more effector devices responsive to the divergence.

### FIELD OF THE INVENTION

This invention relates to medical monitoring and control systems for healthcare.

### BACKGROUND TO THE INVENTION

Today various monitoring devices like ECG, PPG, Blood pressure etc. work independently. Even in a Vital Signs monitor for example, where several traces of measurements of ECG, Pulse Plethysmo-Graphy, IBP, respiration etc. are displayed on the same display, they just share a display but each parameter is treated separately.

Combining signals and data from different sources can be used to create signal redundancy and allows for more reliable and accurate signals and noise reducing.

Certain parameters can be measured in more than one way. For example, heart rate can be measured both from an electrocardiogram (ECG) and from a pulse oximetry waveform (used to calculate oxygen saturation in peripheral blood).

Prior art examples can be found, for example, in U.S. Pat. No. 7,647,185 which discloses a method for combining physiological measurements from two or more independent measurement channels, particularly physiological measurements such as heart rate. In WO 2009125349 a system for measuring blood circulation parameters in a patient, uses multiple sensors including a contact microphone and a pressing element to estimate continuous blood pressure (BP).

In terms of treatment, U.S. Pat. No. 7,278,983 describes methods and apparatuses for calculating and transmitting medication dosage or bolus information are provided, U.S. Pat. No. 7,860,583 describes a system and method of managing therapy provided to patients and WO2006030534 describes a system for non-invasive delivering and activating therapeutic agents within the body.

FIG. 1a shows how medical devices, including sensor devices and effector devices are operated today in a medical environment. In FIG. 1a there are examples of three independent medical devices in use around the patient: a Vital Signs Monitor 100, an Infusion pump 110 and an Ultrasound imaging device 115.

The Vital sign monitor 100 includes a display 102, a set of analog amplification modules 104 like ECG, PPG, IBP and the like. The corresponding analog electrodes 105 connect to each module. E.g. ECG electrodes 108 to the ECG modules, inflatable cuff 107 to the NIBP module, optical photodiodes and LED 106 to the PPG. The Vital signs monitor 100 includes also a user interface that is not depicted in FIG. 1a.

The Infusion pump 110, which is a treatment device, includes also a small display, control unit for controlling the pump, and the pump itself. In addition, it has a User Interface for manual control of the pump by the doctor or nurse.

An imaging device is also shown, in FIG. 1a this is a portable ultrasound imaging device 115. The Ultrasound imaging 115 has its own display 116, a signal processing unit and a beam former that is connected to the ultrasonic probe.

Each one of the many devices around the patient has its own display, processing unit, User Interface etc. There is no communication among them and even inside the Vital Signs Monitor 100, each channel 104 (ECG, NIBP, PPG and the like) works independently.

As will be appreciated, each of these devices is operating independently and there is no mechanism by which data can be shared between such devices. Accordingly there is a desire for a device to be able to provide such features to improve the analysis and control of medical devices.

### SUMMARY OF THE INVENTION

The present invention relates to apparatus that may be used for combining measurements, monitoring, imaging, clinical data including models and effector devices for a patient. A medical console is provided that allows medical devices information to be monitored, combined and controlled from a single central console or server—a Patient Server.

According to a first aspect of the invention there is provided a clinical care system comprising a personal medical console and a plurality of medical devices, the medical devices comprising one or more sensor devices and one or more effector devices, wherein the one or more sensor devices each comprise a sensor configured to sense physiological characteristics, the one or more sensor devices being configured to communicate with the medical console to transmit sensor data derived from the sensors to the medical console; wherein the medical console is connectable to a plurality of different sensor devices and different effector devices and configured to receive sensor data from the one or more sensor devices and output control data defining one or more actions for controlling the one or more of the effector devices to the one or more effector devices, and comprising: an analysis engine configured to process the sensor data received from the plurality of sensor devices and output control data in response to the sensor data, wherein the processing comprises determining a divergence of the sensor data to a target health state, and generating the control data for the one or more effector devices responsive to the divergence; and wherein at least one of the one or more effector devices is configured to receive control data from the medical console to control operation of the respective effector device responsive to the control data.

The personal medical console provides a central resource for managing the care of a patient and as such, may be positioned at a patient's bedside or remotely controlled. One or more sensors are connected to the medical console as well as one or more effective devices (which may be, for example a treatment device used to treat a patient. In the medical console, an analysis engine processes the sensor data received from one or more sensor devices and can generate control data used to control an effector device in response to the sensor data. The analysis engine determines a divergence of the sensor data to a target health state for the patient and then generates the control data for outputting to one or more effector devices in response to this determined divergence. The target health state (a predetermined clinical goal) may comprise one or more different characteristics which may be derived from stored data relating to the patient, data derived from learning an anticipated response to the particular sensor data readings, from predetermined or pre-programmed information configured by for example a medical practitioner. It may be, for example, an intention of keeping the heart rate within a predefined range, or a number of activations of a ventilator per minute. Further ways of determining the clinical goal are may also be possible and the preceding list should not be considered to be limiting of such clinical goal data that may be used. Moreover, such a divergence may vary depending on the condition of a patient and so such data may vary over time. An effector device, for example a treatment device, can then receive such control data from the medical console and control the operation of the device accordingly so that it may counteract any change in condition (or maintain the existing condition). The personal medical console may also be adapted for use by a medical professional at a patient's bedside.

Furthermore, in addition to controlling an effector device, an alert may be generated to a care giver.

Embodiments of the Patient Server may be implemented on a portable computing device, such as a SmartPhone environment, e.g. Android. Such a device, acting as the Patient Server may then interface to existing medical devices like the vital signs sensors, imaging devices, infusion pumps, etc. Although SmartPhone hardware may be used, the cellular may not be necessary in some embodiments (although they can come in handy for tele-health). Moreover, such a platform allows for easier development and use of plug and play type interfaces, such as those discussed later. Examples of effector devices include infusion pumps, ventilators. Devices may be mechanical, energetic (thermal, ultrasonic or electromagnetic radiation including LASER), chemical (e.g. injection by an infusion or injection pump).

The clinical care system may further comprise a plurality of sensor devices. In such a situation the processing may then further comprise determining a secondary physiological characteristic from a combination of the sensor data, in other words the sensor data from the plurality of devices is combined to form a new physiological characteristic. This secondary physiological characteristic may then be used to determine a divergence to the target health state in order to control the one or more effector devices. Such a secondary physiological characteristic may be derived from a combination of measurements from two of the same type of device (they may, for example, both be monitoring heart rate) or may be from different devices each sensing a different physiological characteristic. In the example whereby two devices sense the same characteristic the generated data may be improved (for example a noise filtered version of the original sensor data). Moreover where different physiological characteristics are combined from multiple sensor devices such data again may provide improved (for example filtered/noise reduced signals) or may be used to create new physiological characteristics not individually derivable from the sensor devices in isolation.

The medical console may comprise a historical data store configured to store one or more of the sensor data and the control data. In other words, the data store may store a history of sensor data and/or control data providing a log of the readings from sensors and/or any control data or controlled operation sent to the effected devices in order to ascertain what control information has been sent responsive to any sensed data. This may be used, for example by a medical practitioner to assess the effectiveness of treatment of a patient and to view the history of treatment

The medical console may further comprise a patient data store configured to store patient medical history data which allows direct and instant access to details pertaining to the patient by a medical practitioner when viewing or assessing the medical status of a patient. Furthermore this information can also be used to assess a course of treatment for a patient based upon the information stored therein. In variants however the patient medical history data may be stored remotely, and the medical console may have access to such a remote storage facility in order to deliver the information and present it or use such data.

The target health state may be learnt from one or more, or a combination of stored sensor data, stored control data (in the historical data store), and/or the stored patient medical history data (in the patient data store). In other words such stored data can be used in order to assist or improve in the derivation of a target health state for the patient in question. Such data may be used in isolation, or rely on a medical practitioner's further guidance, (although it will be appreciated that in many situations further input to verify such data may be desired) before a patient is treated.

The analysis engine in the medical console may be configured to merge the one or more of the sensor data from the one or more sensor devices, the control data from the one or more effective devices and a secondary physiological characteristic into a data stream. In other words combinations of the available data relating to sensing and/or control may be merged into a unified data stream allowing such data to be sent to a visualisation engine (which in some embodiments is part of the medical console). This data can then be converted to a graphical data stream for rendering on a graphical display. This allows visualisation of all the information. The data visualised may also be configurable to allow a medical practitioner to view any necessary information they so desire from one or more of the effector devices and any derived data. The medical console may further comprise a graphical display, in particular in integrated graphical display such that such rendered information is presented directly on the medical console. However in variants the medical console may not comprise a display and may be coupled to a separate display unit.

On the display, the initialisation engine may be configured to overlay one or more of the sensor data from the one or more sensors, a secondary physiological characteristic and/or the control data on the graphical display such that this information can be compared in a manner so desired by a medical practitioner.

The medical console may further comprise a device recognition module to detect at least one of the medical devices, and to interrogate the medical device which has been detected to identify the capabilities of the detected medical device. In other words, when a device is plugged into the medical console or wirelessly connected the medical console has the ability to determine what the device is in order to receive and interpret such sensor data appropriately or determine what form of control may be applied to the effector device that has been plugged in. It may also be necessary to download further information to the medical console if it has not been previously aware of such a device. Such information may be derived or obtained from the medical device itself or may require an update to the medical console in order to interpret the information received. Such an update may be downloaded remotely, for example, from a central database.

At least one of the medical devices may be configured to advertise its services to the medical console responsive to being coupled to the medical console. The console is may then be able to recognise what capabilities are provided by the recently connected medical device. Furthermore, a device may also communicate information relating to its configuration, such as its position on a patient, so that the medical console is aware of such information when performing any analysis/processing.

The medical console may be further configured to transmit sensor control data to one of the one or more sensor devices for controlling the one of the one or more sensor devices via the device interface. The one of the one or more sensor devices may then be configured to receive such sensor control data and then process the sensor control data in order that the particular sensor device can be configured. In other words, the medical console may provide capabilities to configure sensor devices, which may include, for example, configuring when measurements may be taken, calibrating such sensor devices, activating or de-activating and the like.

At least one of the one or more effector devices may be configured to transmit effector device status data of the medical console. The medical console may then be further configured to receive the effector devices status data and process the effector devices status data in order that it can monitor the status of the effector devices. The effector devices may than also signal the medical console of any issues, problems or changes in operation that may be of relevance.

The medical console may further comprise a user interface which may be separate, or part of the graphical user interface (i.e. a touch screen for example). The user interface may be used for configuring operation of one or more of the medical console the one or more sensor devices and one or more effector devices. Examples of such interfaces are separate keyboards, touch screen displays (overlaid on the graphical display for example), keyboard, mouse and the like. A remote interface capability may also be provided allowing control (if so desired) from a remote terminal.

The sensor devices may each comprise a sensor interface coupled to the medical console to transmit the sensor data. Thus, the medical console may then further comprise a device interface coupled to each of the sensor interfaces to receive the sensor data from each of the one or more sensor devices. The medical console may then be further coupled to one or more control interfaces on a respective one or more effector devices to transmit the control data to the effector devices. Then, the at least one of the one or more effector devices may be configured to receive the control data from the medical console via a respective control interface.

The device interface, sensor interfaces and/or control interfaces may comprise network interfaces and the medical console may be further coupled to the one or more sensor devices and one or more effector devices by a network connection. In variants however such connections may be USB interfaces for example or any other form of known communication interface. The medical console may accordingly comprise an integrated network/USB hub into the device interface, or may alternatively have one or two (or more) connection points and allow for use of an external network/USB hub.

The medical console may further comprise remote access interface for remotely managing operation of the medical console, the one or more sensor devices and the one or more effector devices. Such a remote access interface may also be used for transmitting or receiving data remotely related to the operation of the medical console. It may also be used to obtain a medical history data for a particular patient and downloaded to the medical console. This may be provided via a network interface, for example.

The sensor interfaces, control interfaces and device interface are preferably wired interfaces although it would be appreciated that wireless interfaces may also be used and the invention as herein described should not be limited to wired interfaces only. Wireless interfaces may be more useful for patients that are able to walk or mobile health applications.

The one or more sensor devices may comprise ECG, non-invasive blood pressure, oxymetry, real time arterial blood gas and imaging devices for example. It will be appreciated however that further sensor devices may be provided and the invention is not limited to use of this list only.

According to a second aspect of the invention there is provided a personal medical console connectable to a plurality of different sensor devices and different effector devices and configured to monitor operation of one or more sensor devices and control operation of one or more effector devices, the one or more sensor devices each comprising a sensor configured to sense physiological characteristics and transmit sensor data derived from the sensors to the medical console; the one or more effector devices each configured to receive control data from the medical console to control operation of the respective effector device responsive to the control data; wherein the medical console is configured to receive sensor data from the one or more sensor devices and output control data defining one or more actions for controlling the one or more of the effector devices to the one or more effector devices, and comprising: an analysis engine configured to process the sensor data received from the one or more sensor devices and output control data in response to the sensor data, wherein the processing comprises determining a divergence of the sensor data to a target health state, and generating the control data for the one or more effector devices responsive to the divergence.

The personal medical console may be further configured to generate an alert event, from an effector or another device responsive to the processed sensor data such that a care giver/doctor/nurse can be alerted if medical care is needed. Such an alert may be local on the medical console, or may be a remote alert, such as to a buzzer, pager, via SMS, or over a network.

The medical console may further comprise a plurality of sensor devices, and wherein the processing further comprises determining a secondary physiological characteristic from a combination of the sensor data, and wherein the determining a divergence comprises a determining a divergence of the secondary physiological characteristic to the target health state (predetermined clinical goal). The personal medical console may also be adapted for use by a medical professional at a patient's bedside.

In other words, sensor data may be read from two or more sensor devices processed to determine a physiological characteristic derived from this combination of data and then processed to determine a divergence of this combined characteristic to target health state as previously discussed.

The plurality of sensor devices may be configured to each sense a different physiological characteristic such that the processing then comprises combining this data related to at least two different physiological characteristics in order to generate the control data that is then used to control an effector device.

The medical console may further comprise a device interface couplable to sensor interfaces on each of the one or more sensor devices to receive the sensor data from each of the one or more sensor devices. The medical console may be couplable to a control interface on each of the one or more effector devices to transmit the control data. Such interfaces may comprise network, USB, wireless interfaces and the like as discussed for the first aspect of the invention.

According to a third aspect of the invention there is provided a personal medical console for managing a plurality of medical devices, the plurality of medical devices comprising a plurality of sensor devices and each comprising a console interface couplable to the medical console and configured to communicate with the medical console to provide medical device data to the medical console pertaining to the operation of the medical devices; the medical console comprising: a device interface couplable to each of the console interfaces on the plurality of medical devices, the device interface configured to receive the medical device data from each of a plurality of the medical devices; an analysis engine coupled to the device interface, the analysis engine configured to process the medical device data and determine a secondary physiological characteristic from a combination of the medical device data, convert the secondary physiological characteristic into a data stream; and a visualisation engine configured to convert the data stream into a visualisation stream for rendering on a display. The personal medical console may also be adapted for use by a medical professional at a patient's bedside.

Data from a plurality of sensor devices may be combined in the medical console by the analysis engine in order to determine a secondary physiological characteristic from a combination of the medical device data. Such data may then be converted into a data stream and sent to a visualisation engine in order to convert the data stream into a visualisation stream for rendering on a display so that the newly generated data can be visualised.

The plurality of medical devices may further comprise one or more effector devices and the medical console may then processing the medical device data in order to determine a divergence of the secondary physiological characteristic to a target health state. Such data may then be used to generate control data for output over the device interface for controlling the one or more effector devices responsive to the divergence. Thus, in addition to viewing the secondary physiological characteristic from a combination of the multiple sensor signals, such data can also be used to control one or more effector devices.

According to a fourth aspect of the invention there is provided a clinical care system comprising the medical console of the third aspect and a plurality of medical devices coupled to the medical console, the plurality of medical devices comprising sensor devices and/or effector devices and each comprising a console interface configured to communicate with the medical console to provide medical device data to the medical console pertaining to the operation of the medical devices.

According to a further aspect of the invention there is provided an interface adapter for interfacing a sensor device to the personal medical console of the above described aspects, the sensor device comprising a sensor to sense a physiological characteristic and a data output to output measurement data, the interface adapter comprising: a data feed input to receive measurement data from the sensor; a sensor device interface to output the sensor data, and wherein the interface adapter is configured to implement a common set of procedures to provide a common data access specification for communicating the sensor data via said sensor device interface.

The interface adaptor provides a common set of procedures that allow the personal medical console to interface with interface adapters, each of which may be connected to different sensors (or in other embodiment—effectors). This allows for a common data access specification standardised across interface adapters compatible with the personal medical console. Such features may include a common memory map for access configuration features within the interface adapter, communication protocols, and the like. The interface adapter may be used to allow a conventional sensor device to be coupled to the personal medical console according to the previously described aspects. Such a sensor device may be used to sense a physiological characteristic and to generate the measurement data from the sensor reading. The interface adaptor converts this measurement data into a suitable format for outputting according to the necessary messaging protocol used by the personal medical console. This may be for example based on a network interface protocol as known in the art, a USB protocol, or any other form of known protocol such as Bluetooth, W-Fi (802.11), Zigbee, NFC, IR. The interface adapter may also be provided as an add on to other device already capable of communicating with the medical console, being implemented as a software extension running on another device.

The interface adaptor may also be used to provide identification data which can be used in order to transmit capability information to a medical console when connected to such a medical console. In other words, this allows plug and play type capabilities enabling a conventional sensor device to be plugged into the above medical console via the interface adaptor. Such an interface adapter may preferably be tailored to a particular sensor device (or configured to support multiple different devices) allowing capabilities of that particular sensor device to be transmitted to the medical console once coupled to the interface adaptor. Furthermore such an interface adaptor may also be able to respond to a request via the sensor interface for such identification data upon which the processing engine within the interface adaptor may be used to read identification data from the data store and output the identification data to the sensor device interface.

Such an interface adaptor may also be provided to an effector device and operate as discussed above.

According to a still further aspect of the invention there is provided a personal medical console for accompanying a patient under medical care and adapted for use by a medical professional, the personal medical console configured to connect to a plurality of medical devices, the medical devices comprising sensor devices and one or more effector devices; wherein the medical console is configured to: analyse a patient health state from sensor data received from the plurality of sensor devices; process the sensor data and generate control data responsive to the processed sensor data for controlling one or more effector devices; and transmit the control data to the one or more effector devices to control operation of the one or more effector devices. Patient state may be analysed from integrated sensor data received from the plurality of sensor devices. Clinical history data may also be incorporated in to an analysis.

According to a still further aspect of the invention there is provided a method of managing a plurality of medical devices using a personal medical console, the medical devices comprising one more sensor devices and one or more effector devices, the method comprising: monitoring operation of one or more sensor devices coupled to the personal medical console, the one or more sensor devices each comprising a sensor configured to sense physiological characteristics and generate sensor data; analysing the sensor data received from the plurality of sensor devices by determining a divergence of the sensor data to a target health state; and outputting control data to one or more effector devices coupled to the medical console responsive to the divergence.

Features of the above described aspects of the medical console may also be applicable to such a method, and may include one or more of the following:

When providing a plurality of the sensor devices, the analysing may further comprise determining a secondary physiological characteristic from a combination of the sensor data, and the determining a divergence may comprise determining a divergence of the secondary physiological characteristic to the target health state.

The method may further comprise detecting a coupled medical device when coupled to the medical console such that a medical console is aware of the newly connected device. This may also comprise broadcasting the presence of a newly connector device. Subsequently, the method may further comprise interrogating the newly connector device in order to identify capabilities of the device. Once features have been identified, which may be from data received from a newly connected device, or by reference to another data source (a data store within a medical console or a remote data store) then if the device is a sensor device, the method may further comprises analysing the received sensor data from the newly detected and installed device.

We further describe a method of treating a patient comprising providing a personal medical console and a plurality of medical devices, the medical devices comprising one or more sensor devices coupled to a patient to sense physiological characteristics and and one or more effector devices which may optionally be coupled to a patient, wherein the one or more sensor devices each comprise a sensor configured to sense physiological characteristics of the patient, the one or more sensor devices being configured to communicate with the medical console to transmit sensor data derived from the sensors to the medical console; wherein the medical console is connectable to a plurality of different sensor devices which may be coupled to the patient and different effector devices and configured to receive sensor data from the one or more sensor devices and output control data defining one or more actions for controlling the one or more of the effector devices to the one or more effector devices, and comprising: an analysis engine configured to process the sensor data received from the plurality of sensor devices and output control data in response to the sensor data, wherein the processing comprises determining a divergence of the sensor data to a target health state, and generating the control data for the one or more effector devices responsive to the divergence; and wherein at least one of the one or more effector devices is configured to receive control data from the medical console to control operation of the respective effector device responsive to the control data.

The processing in the method may further comprise determining a secondary physiological characteristic from a combination of the sensor data from a plurality of sensor devices, and wherein the determining comprises determine a divergence of the secondary physiological characteristic to the target health state.

Features of the personal medical console are applicable to one or more aspects of the inventions.

In addition to the examples described above, sensor data may sensor data may also from a storage device (e.g. ECG Holter or DLNA streamer server). Furthermore, the medical console may also be connect to alert devices such as a buzzer, SMS messages, and other devices such as displays and medicine table dispensers.

### BRIEF DESCRIPTION OF THE DRAWINGS

These and other aspects of the invention will now be described, by way of example only, with reference to the following drawings in which:

FIG. 1a shows an example of the prior art with each medical device working independently;

FIG. 1 b shows an overview of the medical console (Patient Server) according to an embodiment of the invention in which device monitoring and control is combined;

FIG. 2 shows further details of the medical console (Patient Server) of FIG. 1 b;

FIG. 3 shows the medical console (Patient Server) used in an Integrated Clinical Environment (ICE) platform;

FIG. 4 shows the Patient Server as part of a DNLA form of platform;

FIG. 5a shows an example of a DNLA enabled Patent Server for combining BPCG (Blood-Pressure Cardio-Gram) signal and a video camera, displaying on a DNLA enabled TV;

FIG. 5b shows an application of the Patient Server for blood pressure (BP) signals derived from IBP (Invasive Blood Pressure), and activation of a flushing actuator/effector when damping of signal is detected to flush a clogged tube;

FIG. 5c shows use of the arrangement in FIG. 4 on a patient during therapeutic ultrasound treatment of cervical cancer tumor; and

FIG. 6 shows a further example of the Patient Server for combining different types of signals.

### DETAILED DESCRIPTION OF THE PREFERRED EMBODIMENTS

The Patient Server as herein described is an active patient-chart that accompanies the patient, actively collecting relevant clinical information from a plurality of medical devices around the patient. It also analyzes the patient's health state by combining all the information as well as applying effectors to interrogate and treat the patient based on that information. In many examples of the system such control may also be under the caregiver control.

The Patient Server is able to collects information relevant to a patient state (from the sensors for example) in real-time, performs real time analysis of the patient's health state and applies the necessary effectors both to interrogate and treat the patient with the intention of bringing the patient into an improved health state. Such a device may also be under the care giver control.

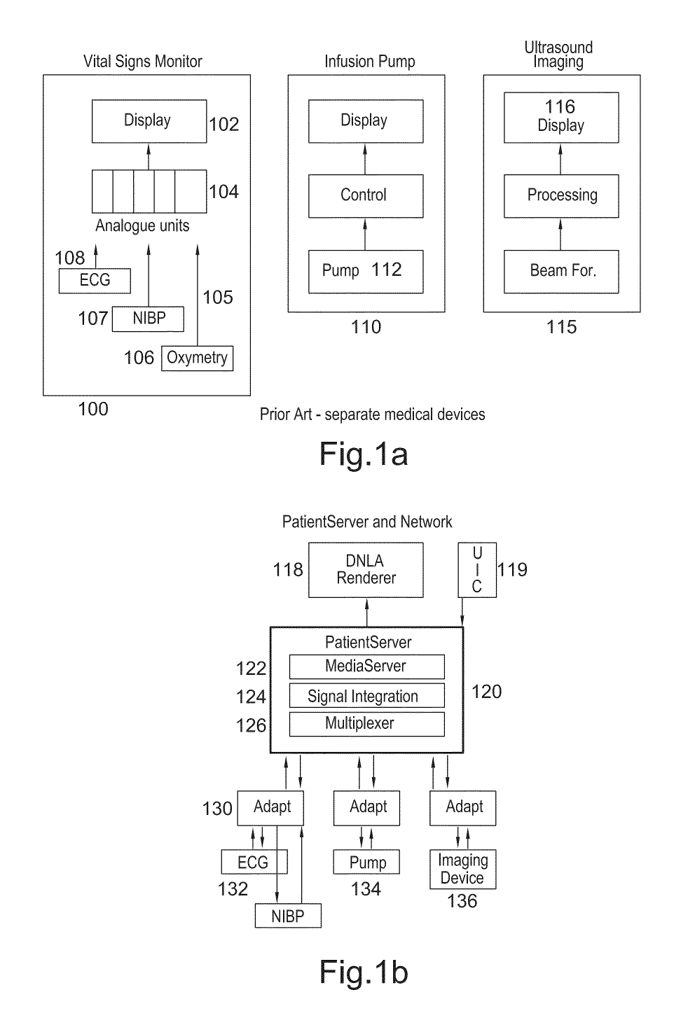

FIG. 1b shows an overview of the Patient Server (medical console) and its associated network of medical devices (sensor devices and effector devices) coupled to the Patient Server. In comparison to the example in FIG. 1a, which shows the existing situation of employing independent medical devices (such as a Vital Signs Monitor 100, an Infusion pump 110 and an Ultrasound imaging 115) all these devices are now coupled to the Patient Server. The Patient Server 120 processes all signals and uses one display 118 and a shared User Interface Controller 119 for controlling, managing and configuring all the medical devices attached. It also allows for storage of a patients medical history including treatment history, sensor history and patient medical records.

Medical devices include, but are not limited to, monitoring devices, medical imaging and drug delivery devices for example. Effector devices may be used for interrogation purposes and/or treatment (for example, in Continuous Non-Invasive Blood pressure monitoring, an effector device may change the pressure level of the sensor.)

The Patient Server 100 may also use plug and play technology (PnP). The Patient Server and devices connected to it adopt standardised protocols for combining medical devices around the patient personal server.

The multiplexer module 136 in the Patient Server communicates with the various devices around the patient. With an embodiment employing UPnP technology or DLNA, it is able to communicate with any Medical device that has a DLNA adapter 130.

DLNA adapter 130 converts such devices to DLNA devices and enables devices to advertise their services as soon as they are in the room and active and make a connection with the Patient Server. For example, an ECG device may advertise that it can provide real-time twelve lead ECG signals (sometimes however only 5 or 3 electrodes are used so it will be designated as 5 lead or 3 Lead ECG respectively); an Infusion Pump may advertise that it may deliver a range of mL/min outputs of a specific combination of therapeutic agents; and an imaging device 136 may advertise its ability to provide a video stream of an ultrasound imaging device.

Signal integration and control module 124 received the multi-channel information from the various devices, using multi-channel models to generate the signals to be displayed and/or employs control algorithms to control effectors like the Infusion Pump. Unlike the regular vital sign monitor 100 as is known in the art that houses independent channels, signal integration in the Patient Server combines/integrates signals from multi sensors and effectors.

The User Interface Controller (UIC) 119 allows the medical staff to interact with the Patient Server and control all the devices through one device (the Patient Server) and one interface. This is in contrast to the situation today, where the Vital signs monitor 100, the infusion pump 110, the Ultrasound imaging 115 and any other device, have their own user interface.

Devices connected to the Patient Server (medical console) may be “plug-and-play” in that when connected to a network they automatically establish working configurations with other devices. Accordingly, monitoring (sensor) devices like ECG and Non-Invasive Blood Pressure 132 (as shown in FIG. 1b), Oximetry, real-time ABG (Arterial Blood Gases), blood and urine analyzers adopting such a protocol can be plugged into or activated in the vicinity of the medical server (Patient Server) and automatically recognised. In some variants, an interface adapter 130 may be used, if for example, an existing, non PnP device is to be attached. The interface adapter then translates the data from the sensor device into an appropriate format for communication to the Patient Server, enabling the medical device to become a medical DNLA device and connected to the Patient Server. With such an adapter 130, existing equipment such as Vital Signs Monitors and medical imaging Ultrasound imaging 136 may be connected. Effectors, such as drug delivery devices like infusion pumps 132 can also be connected through such an interface adapter.

It will be appreciated however that in variants devices may be provided that incorporate the necessary protocol conversion/adapter into the medical device itself, i.e. the Patient Server Network devices are designed as such, and they do not need individual processing units, displays or User Interface leading to cheaper and smaller devices. Adapters can be retrofitted to existing devices.

Clinical data may also be stored on a remote storage server, or within the Patient Server on the storage server—in both cases such data can be retrieved from storage and used.

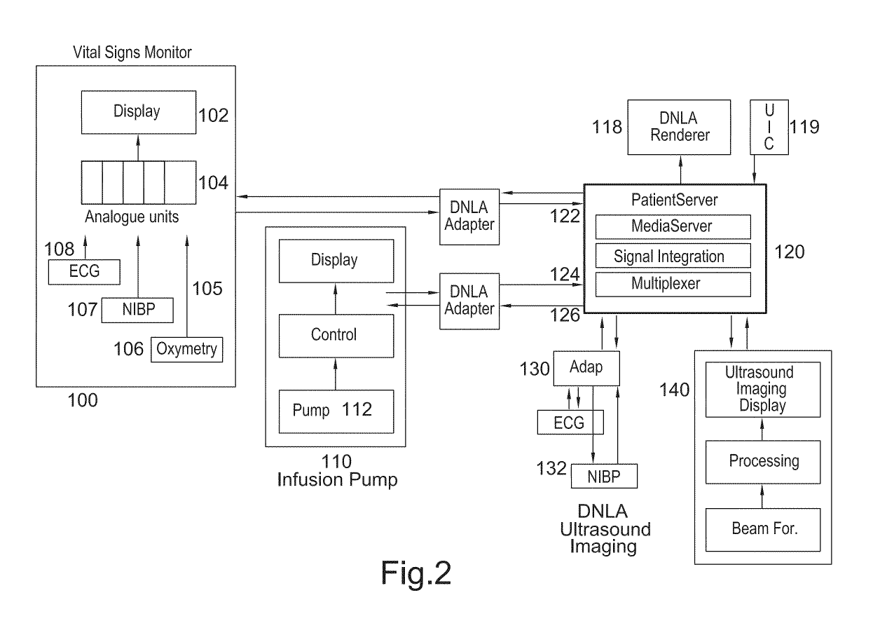

Referring now to FIG. 2, this elaborates on FIG. 1b, and includes an example of an ultrasound imaging display with the DNLA adapter integrated. It also shows the vital sign monitor including several channels, such as ECG, IBP, PPG etc connected to the Patient Server.

The vital signs monitor (VSM) is one device that includes several services designated as 105: ECG (108) signals, IBP (107) signal, PPG (106) signal and Oxygen saturation level etc. When connected through the DLNA adapter, the vital signs monitor device advertises all the services it can provide and expose them to the Patient Server.

In additional to the previous example of sensor devices, it will be appreciated that sensor data may also come from a storage device (e.g. ECG Holter or DLNA streamer server).

The Infusion Pump 110 is connected through a DLNA adapter and also exposes the services it can provide, for example controlling one or more pumps. In addition to exposing the flow rate as any other pump, the Patient Server can also inform about the medications/drugs that are delivered by pump 112.

The Patient Server provides further capabilities. This includes, for example, serving as an “Active Patient Chart”. As such, it combines and records continuously and in real time all the data relevant to the patient clinical status. This is different to current systems where, for example, a regular infusion pump is not “aware” what drugs it delivers. In the present invention, the Patient Server is fed with the pharmaceutical information of the injected drugs, so it may cross correlate the expected effect with the physiological change, as measured by the vital signs and other sensors.

Signal combination can be used to generate additional signals that are not measured directly (e.g. generating central blood pressure signal from peripheral signals). Combination of signals can be used also to create discrete and continuous new parameters and signals such as Cardiac Output. Moreover, any transformation of signals, such as the computation of Cardiac Output from BP waveform may result in a reduction of signal to noise ratio in the signal. This can render the transformation of little or no clinical value and make it prone to critical mistakes in estimation. By combining signals, these limitations may be overcome.

In some instances, adding additional sensing channels not only adds redundancy but can contribute unique and valuable clinical information; For example. while ECG and pulse plethysmography can be used to measure heart-rate, there is additional information in using both simultaneously and combining the signals. Some ECG pulses do not achieve a pressure pulse and detecting this info is clinically significant. i.e. different sensors can add features that are not available from looking at each one separately.

Referring now to FIG. 2, this shows how the Patient Server may use additional sensing channels. E.g. in addition to the Vital signs monitor ECH leads, it can use additional ECG leads from another ECG device. Similarly another NIBP device (e.g. placed on the other hand or ankle) can be also added to the Patient Server network.

To support such capabilities, the Patient Server may be made aware of the sensor location (Brachial, Radial, Ankle for example). By employing this location information, the Patient Server may then calculate, for example, the ABI, which is the index calculated from the difference between the Ankle and Brachial BP. Such a newly calculated value may be considered a secondary physiological characteristic.

Another feature of the Patient Server and network apparatus is its ability to integrate clinical data obtained from a DLNA Ultrasound Imaging device 140.

Today, imaging devices like a portable ultrasound imaging device brought to the patient bed or a device used during treatment or during operation, are not connected in any way to other devices and have their own display and their own user interface.

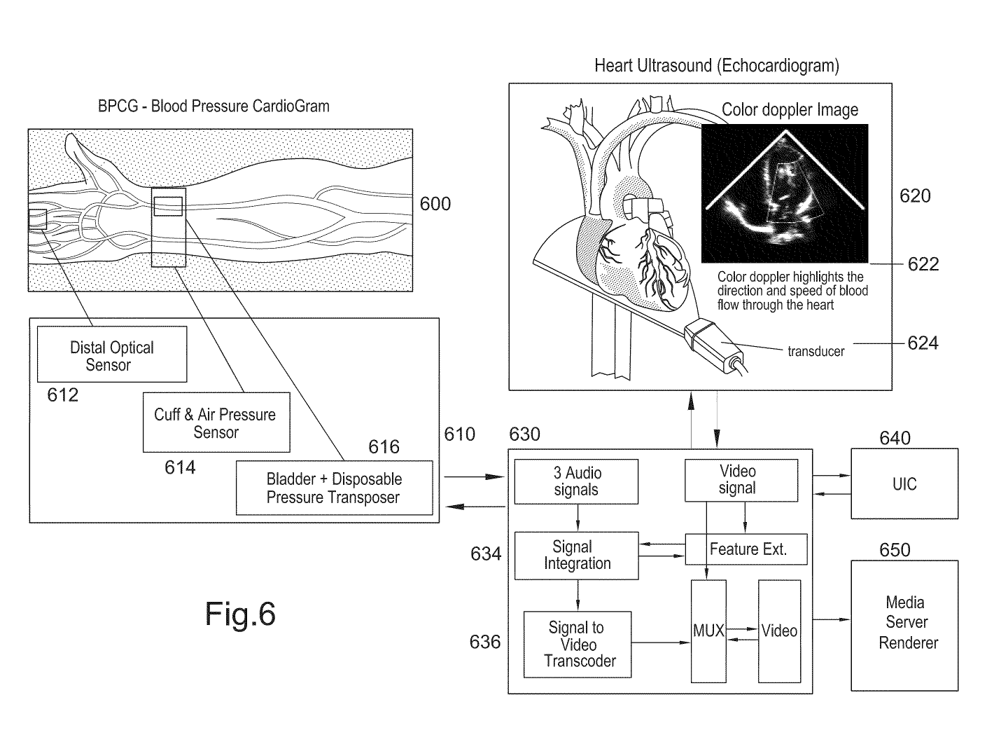

The ultrasound imaging device 140 disclosed herein is connected through a built-in DLNA interface to the Patient Server 120. Using a “Feature Extraction” module inside the “Signal Integration” module 124, the video signal from the imaging can be used to extract continuous cardiac output if the imaging is cross section of a beating heart. As will be explained later with reference to FIG. 6, the same video signal can be used both for displaying the ultrasound image in a window of the DLNA Media Renderer 118, and as a source for a signal that will be combined with PPG 106 and BP 107 signals to compute and display a new signal. Thus, the Ultrasound imaging 140 and signals from the vital signs monitor 100 can be combined.

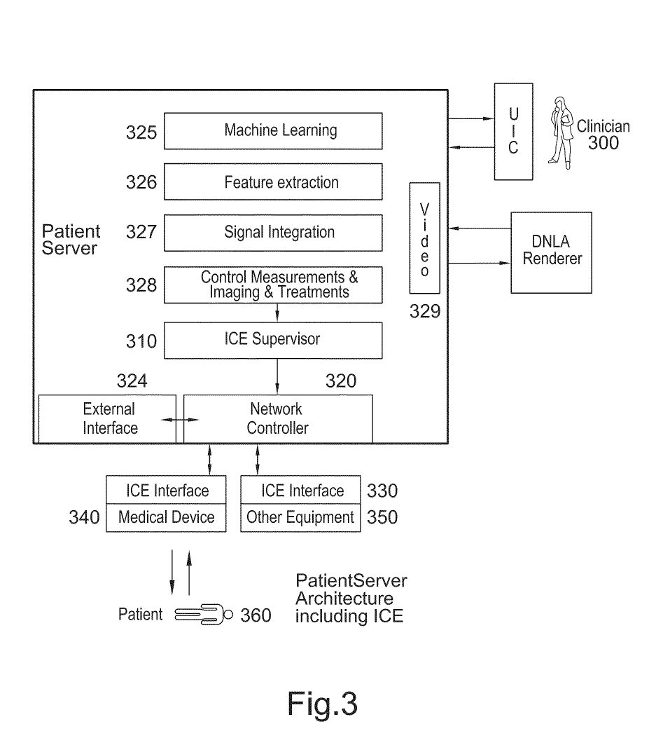

Referring now to FIG. 3, this illustrates Patient Server model and network being used in an Integrated Clinical Environment proposed environment.

The Integrated Clinical Environment (ICE) is a conceptual model of functional elements that has been suggested as an ASTM standard F2761 and is presented in the MD PnP organization website. This standard specifies general requirements, a model and framework for integrating equipment to create an Integrated Clinical Environment (ICE). This standard specifies the characteristics necessary for the safe integration of medical devices and other equipment, via an electronic interface, from different manufacturers into a single medical system for the care of a single high acuity patient. This standard establishes requirements for a medical system that is intended to have greater error resistance and improved patient safety, treatment efficacy and workflow efficiency than can be achieved with independently used medical devices.

ICE is a conceptual suggestion of communication protocols and the Patient Server as herein described can be adapted in some embodiments to employ the ICE communication protocols. In variants other communication protocols can also be used, including proprietary protocols. The Patient Server can provide an “Active Patient Chart” and integrate and control all devices around a specific patient in one Patient Server that accompanies the patient at all times in such a platform.

Realization of the proposed Medical Device plug and play (MD PnP) architecture allows device-to-device networking of Vital Signs Monitors (as rendering devices), networked medical measurement and monitoring devices like ECG, Invasive Blood Pressure Arterial Line, Pulse Oximetry devices, imaging medical devices like medical ultrasound, X-Ray devices, imaging intra-vascular catheters and drug delivery devices like infusion pumps devices. MD PnP as proposed here is a distributed, open architecture protocol based on established standards such as the Internet Protocol Suite (TCP/IP), HTTP, XML and SOAP. MD PnP control points are devices which use MD PnP protocols to control MD PnP devices.

Such capabilities fulfil two requirements for interoperability in medical device systems:

1. Bi-directional data communication capability—Referring to FIG. 2, the Bi-Directional communication supports complete and accurate data acquisition by the Patient Server 120 from vital signs monitors 100, infusion pumps 110, ventilators, portable imaging systems like ultrasound imaging device 140, and other hospital and home-based high-acuity medical devices. Comprehensive data acquisition also enables the development of remote monitoring, advanced clinical decision support systems, intelligent alarms, and robust databases for Continuous Quality Improvement.

2. Medical device control capability to permit the integration—Returning to FIG. 3, the integration of distributed medical devices can be used to provide “error-resistant” systems with safety interlocks between medical devices to decrease use errors, closed-loop systems to regulate the delivery of medication and fluids, and remote patient management to support healthcare efficiency and safety.

Accordingly the use of Patient Server base system in an ICE based platform is able to:
1. Provide a future platform for the development and adoption of open standards and technology to support medical device interoperability. The adoption of safe and effective networked medical device systems requires an ecosystem of ancillary devices and functions. (complying with the ecosystem standard for the Integrated Clinical Environment (ICE), has been published as ASTM F2761-2009), and addresses:
   a. A “network manager” function provided by the Patient Server to support plug-and-play connections of medical devices
   b. Privacy/security/audit trail—to assure confidentiality and reliability of data
   c. Authorization—to prevent non-conforming devices from affecting the network
   d. Clinical/Business rules and rule engine—to support clinical algorithms
   e. “Black-box” recording—of network traffic and other data to support forensic analysis and to detect/prevent system problems.
2. Define a regulatory pathway for the proposed systems in partnership with regulatory agencies. (The FDA has been a key collaborator.)
3. Elicit clinical requirements for proposed interoperable systems to improve safety and efficiency.
4. Use MD PnP simulation tools to:
   evaluate ability of candidate interoperability standards to meet clinical requirements
   model clinical use cases (in simulation environment)
   develop and test related network safety and security systems, especially to enhance the understanding of the technical issues at the intersection of Biomedical Engineering and Information Systems
   support interoperability and conformance testing
   serve as a resource for the medical device interoperability community
   Increase the safety and applicability of proposed solution.

### Application Areas for Networked Medical Device Systems

Networked medical device systems can be used to support improvements in work-flow and reductions in medical errors and healthcare costs to the benefit of patients throughout the continuum of care: from the home, to pre-hospital transport, and to clinical areas as diverse as the OR, ICU, general hospital ward and even outpatient clinics and remote monitoring and treatment in the patient home.

The integration of individual medical devices into patient-centric networked systems can provide real-time comprehensive data for the electronic medical record (EMR) and can support advances in patient safety and work-flow improvements such as:
Clinical decision support
Medical device safety interlocks
Physiologic closed-loop control of medication, fluid delivery, and ventilation
Monitoring of device activity and performance
Automated system readiness assessment (prior to starting invasive clinical procedures)
Support of remote patient management (“e-ICU”)
Safeguarding of protected patient information through real-time encryption
“Plug-and-play” modularity to support “hot swapping” of “best of breed” devices
Facilitation of disaster preparedness: real-time inventory of hospital equipment in-use and national stockpiles, and rapid deployment of devices in makeshift emergency-care settings
Avoidance of unnecessary redundancy by using shared resources
Reduction of the cost and implementation barriers to technology dependent innovation.

The medical world is very conservative and doctors and nurses are extremely busy, so any suggestion for combinations that will require a complicated and time consuming set-up is not going to work. Combination of devices needs to allow any number of devices and data sources as needed for a certain patients, with enough redundancy to compensate for noise and in accuracy due to measurement errors. The devices may be dynamically changed in the process of the diagnosis and treatment, as devices that have little or no contribution might be replaced by more needed devices. The proposed architecture and the proposed integration algorithms will estimate the relative contribution of each device to the overall diagnosis and treatment. Following that, devices that might have small contribution might be removed from the bed of a specific patient, while other devices or information sources might be dynamically added.

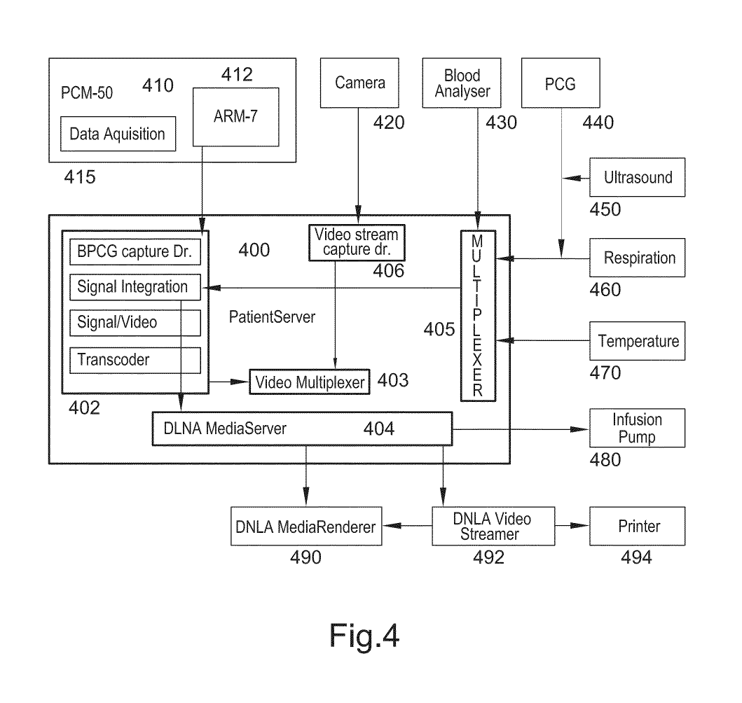

Referring now to FIG. 4, this shows further details of an example embodiment of a Patient Server adopting a MD PnP or DLNA standard.

In FIG. 4 the Patient Server 400 is using the MD PnP or ICE interfaces (not shown in this block diagram) to connect to a multitude of medical and other devices.

The original DLNA standard provides for two types of data streams: Audio (uni-dimensional) and Video (two-dimensional imaging, either stills or video).

A DLNA Media Server Renderer 490 can display video signals, but is unable to display uni-dimensional signals like a regular vital sign monitor can. Accordingly the Patient Server is able to process such signals in order to convert then into an appropriate format for visualisation. In order for the Patient Server 400 to be able to use a DLNA Media Renderer (E.g. DLNA TV), the signals may be converted to the video stream that a device such as a DLNA Media Renderer 490 can handle.

In FIG. 4, “PCM-50” 415, which is a medical device made by CardioScientific, is a monitoring device for non-invasive continuous BP. “PCM-50” 415 performs data acquisition through the data acquisition module 410 from three sensors placed on the patient hand as shown in FIG. 6 below. An ARM-7 processor 412 is embedded in the device and communicates with the Patient Server 400. The ARM-7 processing module 412 gets the three channels from the Data Acquisition module 410. The three channels are fed into a multi-channel algorithm that produces one channel of uni-dimensional continuous BP signal referred to as BPCG (Blood Pressure Cardio Gram).

Inside the Patient Server there is a bank of modules, herein generally referred to as an analysis engine 402 that performs the signal processing, and other related features. The first module that is shown in the bank of modules 402 is a driver that captures the uni-dimensional channel of the BPCFG as an “Audio” signal. This BPCG signal is fed together with other uni-dimensional signals that are coming from the multiplexer 405 depicted on the right side within the Patient Server. Such channels can be respiration 460, temperature 470, Phono Cardio Gram (PCG) 440, etc. In principle, any number of channels that are relevant can be added as the need for them arises. Other examples can be ECG channel, Cardiac Output channel, and even channels from devices that measure blood rheology parameters like haemoglobin, blood viscosity, or blood analytes. etc.

In addition to uni-dimensional signals, two dimensional signals such as imaging coming from an Ultrasound Imaging device 450 can be sent as well as a video signal. As will be explained later in FIG. 6, the video signal coming from Ultrasound Imaging 450 can be used both as a video window on the DLNA Media Renderer 490 or analyzed by the Signal Processing 402 to extract a Cardiac Output related signal. This signal can be integrated with the BPCG signal to yield a more accurate and reliable uni-dimensional signal of Cardiac Output, to be displayed on DLNA Media Renderer 490. However, since the DLNA Media Renderer 490 can handle only video streams, the DLNA MediaServer module 404 needs to prepare such a stream.

In addition, the Patient Server can connect to many other devices around the patient, including non-medical devices like a DLNA Digital camera 420, a DLNA TV 490, DLNA Video streamer 492 and/or a DLNA printer 494. This can be very useful for using the Patient Server in the home environment, where the TV and its remote controller can become the main interface for the home based patient.

An advantages of the Patient Server and its network architecture, is that the network can grow as needed adding devices like Blood Analyzer 430, PhonoCardioGram 440 for heart sounds, Respiration 460, Temperature 470, and any other existing devices or even future devices of continuous non-invasive blood rheology measurements like Glucose, Hemoglobin, PTT, etc.

This is in contrast to existing vital signs monitors that are pre-designed to show specific signals like ECG, PPG, respiration, IBP, and temperature—i.e. are hardwired and limited to processing and reporting signals independently to one another, but on a shared display. Such a device is incompatibly with other sensors that exist today or will exist in the future.

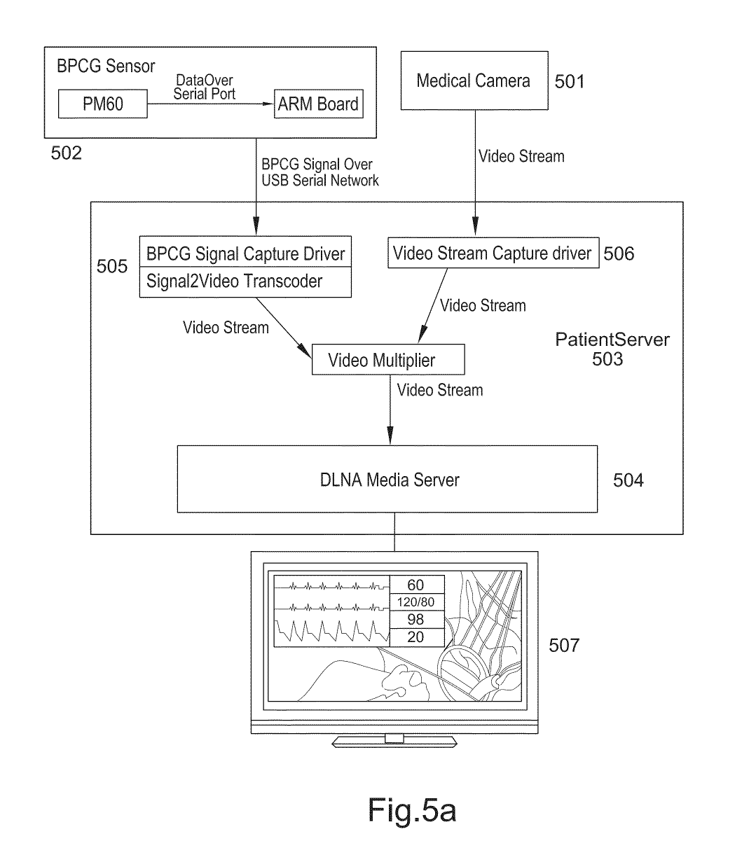

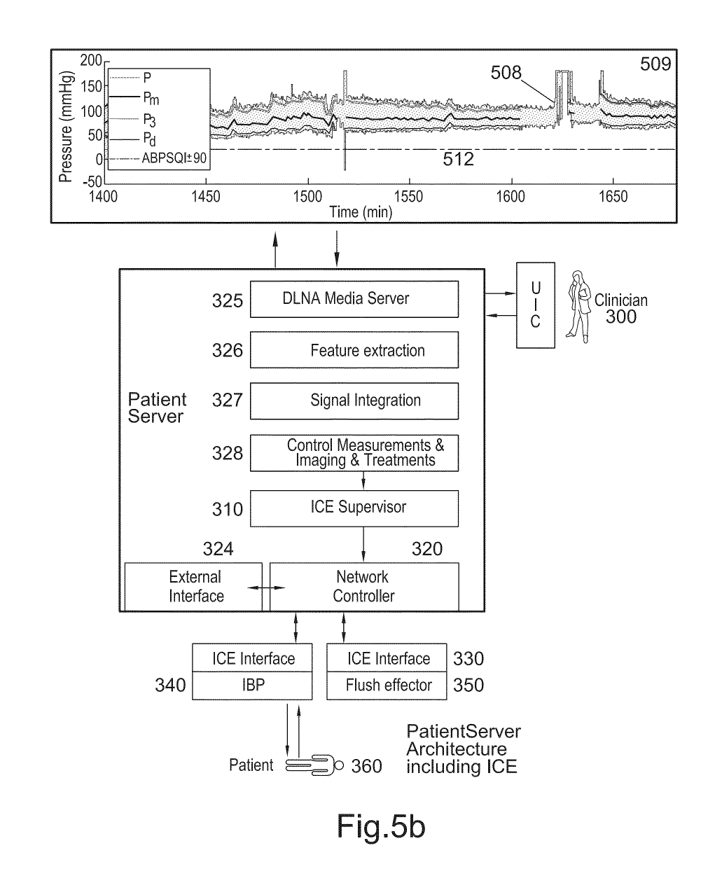

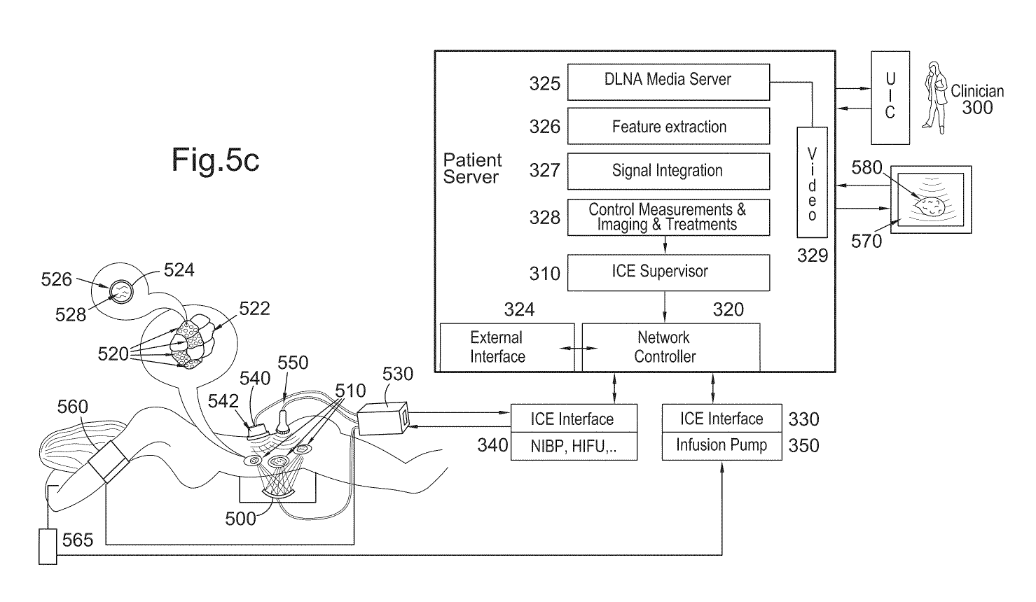

As previously discussed, the Patient Server is not limited to measurements and monitoring, and can take active role in controlling treatments through use of effector devices including drug delivery through infusion pump 480 or administrating other treatments like focused ultrasound energy as described in FIG. 5c. The generation of control data to control such effector devices may be based on stored information within the medical console in the form of desired clinical parameters (for example, a desired heart rate range, ventilation frequency). Such information (a target health state/predetermined clinical goal for the particular effector device(s)) can be used in conjunction with the sensor data in order to adjust the control data output in order to ensure equipment is being controlled appropriately. Data used to derive the control data output may include data derived from the sensor data and/or from sensor signals combined in the medical console and may also incorporate further data from medical records and/or a history of previous sensor/effector control data.

Referring now to FIG. 5a, this shows an implementation of the Patient Server apparatus, connected to a network of three devices. This example demonstrates the handling of different types of signals in the DLNA framework. It illustrates the need for dealing with different types of data streams, namely uni-dimensional signals that look like Audio, and two dimensional signals like video. For Audio, the DLNA supports the simple LPCM (Linear Pulse Code Modulation) format (LPCM is the method of encoding generally used for uncompressed audio), which is used here for the biological signals. For video the DLNA standards support many video formats like MPEG-1 or others. For still images JPEG and similar formats are supported.

The first device is a device that records non-invasive continuous BP signal and is called BPCG sensor 502. The three sensors and their placement on the patient hand are explained later with reference to FIG. 6. The BPCG (Blood Pressure Cardio Gram) device communicates with the Patient Server 503. The module 505 inside the Patient Server captures the uni-dimensional signals and performs the conversion or transcoding into a video signal.

In parallel, the DLNA video camera 501 communicates with the Patient Server 503. The video signal from camera 501 is captured by the module 506 inside the Patient Server.

The two video signals, from modules 505 and 506 are sent to another module which is a video multiplexer, and from there to the DLNA Media Server module 504.

Once the signal is converted to a video stream compatible with DLNA standard for the DLNA Media Renderer 507, it can be displayed on its screen. The display includes both the Uni-dimensional vital signs monitoring signals, like the BPCG, and a video display, as can be seen in FIG. 5a.

Referring now to FIG. 5b, this shows an example use of the Patient Server functionality for combining sensors and effector to maintain accurate and reliable monitoring of Invasive Blood Pressure (IBP).

As can be seen in the DLNA Media Renderer 509, several signals related to the continuous BP recording are presented. The signals depicted are the Systolic BP, Mean Arterial Pressure and Diastolic BP.

The line “ABPSQI” 512 is a computed highlighter that indicates segments where the Arterial BP Signal Quality Index is higher or equal to 90 (on a scale 1 to 100 arbitrary units).

In this case, the Patient Server receives the IBP signal 340 through an ICE interface 330 and communicates it through the ICE Network Controller 320 and ICE supervisor 310 modules inside the Patient Server the Control unit 328.

The Feature Extraction module 326 performs peak detection for each heart beat pulse, and returns the values of systolic, mean arterial pressure and diastolic BP for each BP pulse. In addition, the feature Extraction module calculates the Signal Quality Index for each pulse.

These values are transferred to the Signal Integration module 327 that prepares the signals to be transcoded into video and prepared to be presented by the DLNA Media server 325. Then it will be communicated to the DLNA Media Renderer 509.

The DLNA Media Server may be a service that runs on the Patient Server main processor. However, for clarity, FIG. 5 b shows the DLNA Media Server 325 drawn separately to emphasize the ability of the Patient Server to communicate with a DLNA Media Renderer like a DLNA TV, and that the Patient Server channels all outputs to the user through one display.

The Signal Integration module 327 acts as an analysis engine and analyzes the signals for display from a clinical point of view. As one might notice from the displayed signal, there is a trend from the 1520 min to 1620 min of decrease in Systolic BP and increase in Diastolic BP. This phenomenon is normally not a result of a true change in the BP, but an artefact created by damping in the Arterial-Line tube. This is a common problem that leads to about 100 minutes of incorrect measurement of BP and might lead to wrong clinical decision, unless corrected by flushing the tube with saline.

The Patient Server is able to detect such a problem using the Signal Integration module, and can then issue a command to an automatic flushing effector. However, since flushing with saline may also clinical side effects like blanching (that can be detected by the Oxymetry sensor on the same hand and distal to A-Line insertion point), it may be necessary to get the Physician approval before activating the flushing. This is an example how several sensors and effector can work in concert to improve patient safety and improve quality of monitoring.

Referring now to FIG. 5c, this shows the integration of many sensors and effectors for a closed loop monitoring and treatment of a patient using the Patient Server.

In this example of targeted drug delivery for treating a cancer tumor using High Intensity Focused Ultrasound (HIFU) and Micro-bubbles injection, all the sensors and effectors may work in concert providing an example of the Patient Server and Network functionality.

FIG. 5c shows a female patient suffering from cancer that has been spread and has formed several tumors. The desire is to destroy the tumors without a big abdominal surgery and ablate the cancerous cells while damaging as little as possible healthy cells and tissues. Another problem that needs to be taken care of is that cancer cells can spread during the surgery and also to ensure complete removal of cancerous cells. One approach that has been suggested is to use HIFU to destroy the tumors main mass, while using guiding ultrasound imaging to monitor the treatment, something that has previously been done for prostate cancer and uterine fibroids. However, in addition during the HIFU radiation of the tumors, micro-bubbles that contain toxic agent to the tumor may be delivered to the blood stream by Infusion Pump 565. The micro-bubbles 524 are delivered systemically both by Infusion Pump 565 and through Transdermal ultrasonic enhanced drug delivery through the skin 542. In the presence of an HIFU beam 500 that ablates the tumors they will explode releasing their lethal (to cancer cells) or therapeutic payload into the vicinity of the tumor. This produces a targeted drug delivery that will lead to much higher concentration of the toxic or therapeutic agents in the tumors vicinity. The combination of the focused HIFU 500 and the targeted drug delivery to the tumors will increase the probability of complete eradiation of the cancerous cells.

The imaging ultrasound probe 550 is used for locating the tumors and assessing the temperature change due to the HIFU beam ablation. In addition, micro-bubbles are visible to ultrasound imaging, so the disappearance of the bubbles is another indication to the positioning of the beam and evidence to the cavitation that disrupts the micro-bubbles envelope 526 releasing the toxic or therapeutically payload 528. Knowing the density of micro-bubbles in the coming stream and in the leaving stream can help estimate the dosage released in the tumor vicinity. The cessation of blood flow inside the tumor can be another indication to a successful ablation that does not release cancerous cells to the blood stream.

The synchronization and positioning of the sensors and effectors, as well as close monitoring of the effect, is used for real time adjustment and changing parameters as a result. This combination of measurement, monitoring and treatment control is achieved by the Patient Server.

The Patient Server and its network can be used for realizing such a complex clinical process. The signals from sensors like NIBP 560 and other vital signs (not shown in this FIG. 5c., as well as the Ultrasound Imaging from 550 are communicated via ICE interface 330 into the Network Controller 320 inside the Patient Server. The signals are sent to the Signal Integration 327 for processing and the Feature Extraction module helps calculate positioning and estimated drug dosage in the tumor vicinity. HIFU is used, for example, with MRI device for imaging the temperature change in the target tissues and controlling the beam focus transducer 500. In principle, the MRI can also be included in the loop through suitable ICE interface.

The Clinician 300 may interact with the system through the User Interface Controller as well as view the DLNA Media Renderer 570 that displays the tumor image and microbubbles as detected by ultrasound imaging probe 550. 530 is the beam-former for the Ultrasound imaging and also hub for the various signals.

FIG. 6 illustrates an example of how the Patient Server can be used to handle various types of input signals types, like uni-dimensional signals used in vital signs monitoring, and two-dimensional imaging signals.

This approach can be easily extended into 3-D imaging and 4-D imaging (3-D imaging with a 4^{th} dimension of time.)

In FIG. 6, the data acquisition 610 takes place outside the Patient Server 630, while the integration is done within the Patient Server 630.

FIG. 6 shows also the placement of several sensors and effector on a patient arm. By combining the effector and the sensors the apparatus can arrive at a more accurate, robust to noise and continuous measurement of BP.

The Distal Optical Sensor 612 is in this example an optical PPG sensor. The cuff 614 is an effector that exerts pressure by inflating the cuff 614 and pressing the fluid filled bladder 616 against the Radial artery. The bladder 616 is communicating through a fluid filled tube with pressure sensor that measures the cuff pressure, as well as sensing the pulsation of the BP in the radial Artery.

The three sensors (Distal Optical 612, Air pressure 614 and Fluid pressure in the bladder 616) are communicated to the Patient Server as a multi-channel signal. As the DLNA model deals with Audio signals, it will be sent as a 3 channel Audio signal to the Patient Server 630.

Inside Patient Server 630, module 634 performing the signal processing may use a multi-channel prediction model algorithms to produce one BPCG (Blood Pressure Cardio Gram) (an example of a secondary physiological characteristic) from the three signals.

The signal integration module 634 will also send to the BPCG data acquisition 610 a control signal to control the effector which is in this case an inflated air cuff.

The BPCG signal may then be further processed inside the signal processing module 634, to produce a Cardiac-Output signal from the integration of the area under the BP signal pulses.

In parallel to the processing of the Uni-dimensional signals, image processing of the video signal coming from the Ultrasound Imaging of the heart may be recorded by the Ultrasound Imaging device 620.

Ultrasonic probe 624 and the beam former and signal processor inside Ultrasound imaging 620 (not shown in FIG. 6) produce an ultrasound imaging video signal of a cross section through the beating heart as depicted in FIG. 6. This video signal is communicated as a video stream to the Patient Server. If the Ultrasound Imaging device has also the colour Doppler feature as shown in FIG. 6, additional information conveyed in the colour doppler indicates the direction and velocity of the blood flow through the heart.

Once the Patient Server 630 receives the video signal it is transferred inside the Patient Server into two internal modules. One is the Video multiplexer that will allow the display of the Ultrasound Imaging signal as is by the Media Renderer 650. The other is the Feature Extraction module. From this video image showing the cross section of the left ventricle of the heart, the left ventricle volume can be estimated during end diastole and end systole. The difference in volume between the end diastolic phase image and the end systolic volume (indicated by the dichrotic notch) can be an estimate of the Stroke Volume (SV). From the SV, when multiplied by Heart Rate, the Cardiac Output can be estimated for each heart beat.

By combining the Cardiac Output signal generated from the three uni-dimensional signal from data acquisition box 610 and the Cardiac Output generated from the video signal of the Ultrasound Imaging device 620, a more accurate and robust estimate of the Cardiac Output uni-dimensional signal can be produced. This signal can later be converted to video by the Signal3video Transcoder and sent to the MUX to be displayed by the Media Renderer 650.

The selection of what will be displayed on the Media Renderer Display 650 at any moment may be determined by the user. The user interacts with the Patient Server 610 through the User Interface Controller 640.

Further details on the Medical Plug and Play (MD PnP) are provided below:

MD PnP architecture or DLNA architecture supports Zero Configuration Networking—all networking is done automatically on the fly. A MD PnP compatible device can dynamically join a network, obtain an IP address, announce its name, convey its capabilities upon request, and learn about the presence and capabilities of other devices. Dynamically Host Configuration Protocol (DHCP) and Domain Name System (DNS) servers are optional and are only used if they are available on the network.

Other MD PnP features that are employed in this disclosure include:

Media and device independence—MD PnP technology can run on many media that support IP including Ethernet, Firewire, IrDA, home wiring (ethernet over powerline) and RF (Bluetooth, WiFi, Zigbee, etc.). No special device driver support is necessary; common network protocols are used instead. In addition, there is Operating System and programming language independence—Any operating system and any programming language can be used to build MD PnP products. MD PnP does not specify or constrain the design of an API for applications running on control points; OS vendors may create APIs that suit their customers needs. This is a big advantage for the hospital and tele-health needs, that regardless of the used communication medium, devices can connect automatically and seamlessly.

User Interface (UI) Control—MD PnP architecture enables devices to present a user interface through a web browser. This is, again, a big advantage as today each medical device has its own display configuration and user interface. Today, when a new device is introduced, the medical staff need to learn a new user interface and display. The disclosed invention introduces a unified User Interface for all medical devices.

Programmatic control—MD PnP architecture also enables conventional application programmatic control. This is a major advantage as the combination of measurements, monitoring, imaging, clinical data and treatment benefits from the fact that control of one device can be a function of other devices. E.g. drug delivery dosage and timing can be set as a function of monitoring certain physiological parameters.

Extensibility—Each MD PnP product can have device-specific services layered on top of the basic architecture. In addition to combining services defined by MD PnP Forum in various ways, vendors can define their own device and service types, and can extend standard devices and services with vendor-defined actions, state variables, data structure elements, and variable values. This is a business advantage of the proposed architecture, as various vendors can cooperate on the communication and MD PnP architecture, but still express their unique advantages and contributions. The foundation for MD PnP networking is IP addressing. Each device may implement a DHCP client and search for a DHCP server when the device is first connected to the network. If no DHCP server is available, the device may assign itself an address. The process by which a MD PnP device assigns itself an address is known within the MD PnP Device Architecture as AutoIP. If during the DHCP transaction, the device obtains a domain name, for example, through a DNS server or via DNS forwarding, the device should use that name in subsequent network operations; otherwise, the device should use its IP address. Today, when the address space has been increased to 128 bits, there is no problem that each device will have its own IP static address.

Discovery—Given an IP address, the first step in MD PnP networking is discovery. The MD PnP discovery protocol is known as the Simple Service Discovery Protocol (SSDP). When a device is added to the network, SSDP allows that device to advertise its services to control points on the network. Similarly, when a control point is added to the network, SSDP allows that control point to search for devices of interest on the network. The fundamental exchange in both cases is a discovery message containing a few essential specifics about the device or one of its services, for example, its type, identifier, and a pointer to more detailed information. E.g. when a new Patient Server is added to the Ward Server, it immediately becomes available on the hospital ward network. In the same way, each medical device for monitoring, imaging or drug delivery that is connected to the patient's network, becomes available and can provides services like sending recorded stream of a physiological parameter or being controlled from the Patient Server.

Description—After a control point has discovered a new medical device, the control point still knows very little about the device. For the control point to learn more about the device and its capabilities, or to interact with the device, the control point may retrieve the device's description from the URL provided by the device in the discovery message. The MD PnP description for a device can be expressed in XML and includes vendor-specific, manufacturer information like the model name and number, serial number, manufacturer name, URLs to vendor-specific web sites, etc. The description also includes a list of any embedded devices or services, as well as URLs for control, eventing, and presentation. For each service, the description includes a list of the commands, or actions, to which the service responds, and parameters, or arguments, for each action; the description for a service also includes a list of variables; these variables model the state of the service at run-time, and are described in terms of their data type, range, and event characteristics. One medical device can provide several services. E.g. a monitoring device can include both Blood Pressure, Oxymetry and ECG services.

Control—Having retrieved a description of the device, the control point can send actions to a device's service. To do this, a control point sends a suitable control message to the control URL for the service (provided in the device description). Control messages are also expressed in XML using the Simple Object Access Protocol (SOAP). Much like function calls in computer programs, the service returns any action-specific values in response to the control message. The effects of the action, if any, are modeled by changes in the variables that describe the run-time state of the service.

Event notification—An additional capability of MD PnP networking is event notification or eventing. An MD PnP description for a service includes a list of actions the service responds to and a list of variables that model the state of the service at run time. The service publishes updates when these variables change, and a control point may subscribe to receive this information. The service publishes updates by sending event messages. Event messages contain the names of one or more state variables and the current value of those variables. A special initial event message is sent when a control point first subscribes; this event message contains the names and values for all evented variables and allows the subscriber to initialize its model of the state of the service. To support scenarios with multiple control points, eventing is designed to keep all control points equally informed about the effects of any action. Therefore, all subscribers are sent all event messages, subscribers receive event messages for all “evented” variables that have changed, and event messages are sent no matter why the state variable changed (either in response to a requested action or because the state the service is modeling changed).

Presentation—The final step in MD PnP networking is presentation. If a device has a URL for presentation, then the control point can retrieve a page from this URL, load the page into a web browser and depending on the capabilities of the page, and allow a user to control the device and/or view device status.

Example of MD PnP components include:
MD PnP MedicalServer DCP—This will typically be the hospital ward server, in the ward nurse station, that includes all clinical information of the patients files. This MD PnP-server can provide medical data like the data contained in patients files, and streams it whenever it is asked for it. Patient files including medical signals like ECG/video from medical imaging like Ultrasound, CT, MRI/picture like X-Rays/text files like clinical history, results of lab tests etc. It will send it to MD PnP-clients on the network. In a hospital, this server may typically be in the ward nurse-station. In Tele-health it can be located in the tele-health regional center. A server is generally not interacted with directly, it stores medical data and exposes it via MD PnP. Content can be added or removed remotely by the patients' servers or by the staff personal control Point servers.
MD PnP MedicalServer ControlPoint—which is the MD PnP-client that can auto-detect MD PnP-servers on the network to browse and stream media/data-files from them. This is the UI that the User selects and controls what data to view. Players need not advertise themselves as a MD PnP service, since they are ‘active’ only.
MD PnP MediaRenderer DCP—In our case it is the Vital Signs Monitor that displays the content sent to it by the Patient Server. It is a device that can render (play) content. This is a player that may not need a UI that allows playback from its media player to be controlled by a 3rd device (control point).
MD PnP RenderingControl DCP—control MediaRenderer settings; volume, brightness, RGB, sharpness, and more).

The protocol service used to control playback from the server to the player. Basically it gets a URL exposed in the server and tells the renderer to GET that URL.
MD PnP Remote User Interface (RUI) client/server—which sends/receives control-commands between the MD PnP-client and MD PnP-server over network, (like record, schedule, play, pause, stop, etc.). Implementation of this MD PnP service on the Patient Server is very important for our proposed system, as the Graphic User Interface (GUI) is the most important feature for the medical staff. In our case, providing an RUI service on the Patient Server, allows to use a dumb display as a Medical Rendering device (Vital Signs Monitor).

The key to any MD PnP device is what services it exposes. One MD PnP device can be all or none of the above or any combo as well. Also custom services are often present.

The hard part in creating a real product here will likely be the UI, to support streaming of ECG, video formats & images etc in a way that is a good experience to the user.

No doubt many other effective alternatives will occur to the skilled person. It will be understood that the invention is not limited to the described embodiments and encompasses modification apparent to this skilled in the art lying within the spirit and scope of the claims appended hereto.

1. A clinical care system comprising a personal medical console and a plurality of medical devices, the medical devices comprising one or more sensor devices and one or more effector devices,
wherein the one or more sensor devices each comprise a sensor configured to sense physiological characteristics, the one or more sensor devices being configured to communicate with the medical console to transmit sensor data derived from the sensors to the medical console;
wherein the medical console is connectable to a plurality of different sensor devices and different effector devices and configured to receive sensor data from the one or more sensor devices and output control data defining one or more actions for controlling the one or more of the effector devices to the one or more effector devices, and comprising:
an analysis engine configured to process the sensor data received from the plurality of sensor devices and output control data in response to the sensor data, wherein the processing comprises determining a divergence of the sensor data to a target health state, and generating the control data for the one or more effector devices responsive to the divergence; and
wherein at least one of the one or more effector devices is configured to receive control data from the medical console to control operation of the respective effector device responsive to the control data.

2. A clinical care system as claimed in claim 1, further comprising a plurality of the sensor devices, and wherein the processing further comprises determining a secondary physiological characteristic from a combination of the sensor data, and wherein the determining a divergence comprises determining a divergence of the secondary physiological characteristic to the target health state.

3. A clinical care system as claimed in claim 2, wherein the at least two of the plurality of sensor devices are configured to each sense a different physiological characteristic and the processing comprises combining the sensor data pertaining to the at least two different physiological characteristics to generate the control data.

4. A clinical care system as claimed in claim 1, wherein the medical console further comprises a historical data store configured to store one or more of the sensor data and the control data.

5. A clinical care system as claimed in claim 1, wherein the medical console further comprises a patient data store configured to store patient medical history data.

6. A clinical care system as claimed in claim 4, wherein the analysis engine is further configured to perform processing comprising learning the target health state using one or more of the stored sensor data and stored control data in the historical data store.

7. A clinical care system as claimed in claim 5, wherein the analysis engine is further configured to perform processing comprising learning the target health state using the stored patient medical history data in the patient data store.

8. A clinical care system as claimed in claim 1, wherein the analysis engine is configured to merge the one or more of the sensor data from the one or more sensor devices, the control data from the one or more effector devices and the secondary physiological characteristic into a data stream.

9. A clinical care system as claimed in claim 8, wherein the medical console further comprises a visualisation engine configured to convert the data stream to a graphical data stream for rendering on a graphical display.

10. A clinical care system as claimed in claim 9, wherein the medical console further comprises the graphical display.

11. A clinical care system as claimed in claim 9, wherein the visualisation engine is configured to overlay one or more of the sensor data from the one or more sensors, the secondary physiological characteristic and/or the control data on the graphical display.

12. A clinical care system as claimed in claim 1, wherein the medical console further comprises a device recognition module to detect at least one of the medical devices, and to interrogate the detected medical device to identify capabilities of the detected medical device.

13. A clinical care system as claimed in claim 1, wherein at least one of the medical devices is configured to advertise its services to the medical console responsive to being coupled to the medical console.

14. A clinical care system as claimed in claim 1, wherein the medical console is further configured to transmit sensor control data to one or more of the plurality of sensor devices for controlling the one of more of the plurality of sensor devices, and wherein the one or more of the plurality of sensor devices is configured to receive the sensor control data pertaining the one or more of the plurality of sensor devices and process the sensor control data to configure operation thereof.

15. A clinical care system as claimed in claim 8, wherein at least one of the one or more effector devices is configured to transmit effector device status data to the medical console, and wherein the medical console is further configured to receive the effector device status data and process the effector device status data.

16. A clinical care system as claimed in claim 15, wherein the analysis engine is further configured to merge the effector device status data into the data stream.

17. A clinical care system as claimed in claim 1, wherein the medical console further comprises a user interface for configuring operation of one or more of the medical console, the plurality of sensor devices and the one or more effector devices.

18. A clinical care system as claimed in claim 1,
wherein the sensor devices each comprise a sensor interface coupled to the medical console to transmit the sensor data;
wherein the medical console further comprises a device interface coupled to each of the sensor interfaces to receive the sensor data from each of the one or more sensor devices and further coupled to one or more control interfaces on a respective one or more effector devices to transmit the control data; and
wherein the at least one of the one or more effector devices is configured to receive the control data from the medical console via a respective control interface.

19. A clinical care system as claimed in claim 18, wherein the device interface, sensor interfaces and control interface comprise network interfaces and the medical console is coupled to the plurality of sensor devices and one or more effector devices by a network connection.

20. A clinical care system as claimed in claim 1, wherein one or more of the sensor interfaces, control interfaces and device interface are wireless interfaces.

21. A clinical care system as claimed in claim 1, wherein the medical console comprises a remote access interface for remotely managing operation of one or more of the medical console, the plurality of sensor devices and the one or more effector devices.

22. A clinical care system as claimed in claim 1, wherein the plurality of sensor devices comprise at least two of ECG, non-invasive blood pressure, oximetry, real-time arterial blood gas and imaging devices.

23. A clinical care system as claimed in claim 1, wherein the one or more effector devices comprise at least one of an infusion pump and ventilator.

24. A personal medical console for monitoring the operation of one or more sensor devices and controlling the operation of one or more effector devices,
the one or more sensor devices each comprising a sensor configured to sense physiological characteristics and transmit sensor data derived from the sensors to the medical console;
the one or more effector devices each configured to receive control data from the medical console to control operation of the respective effector device responsive to the control data;
wherein the medical console is configured to receive sensor data from the one or more sensor devices and output control data defining one or more actions for controlling the one or more of the effector devices to the one or more effector devices, and comprising:
an analysis engine configured to process the sensor data received from the one or more sensor devices and output control data in response to the sensor data, wherein the processing comprises determining a divergence of the sensor data to a target health state, and generating the control data for the one or more effector devices responsive to the divergence.

25. A personal medical console as claimed in claim 24, further comprising a plurality of the sensor devices, and wherein the processing further comprises determining a secondary physiological characteristic from a combination of the sensor data, and wherein the determining a divergence comprises determining a divergence of the secondary physiological characteristic to the target health state.

26. A personal medical console as claimed in claim 25, wherein at least two of the plurality of sensor devices are configured to each sense a different physiological characteristic and the processing comprises combining the sensor data pertaining to the at least two different physiological characteristics to generate the control data.

27. A personal medical console as claimed in claim 26,
wherein the medical console further comprises a device interface couplable to sensor interfaces on each of the one or more sensor devices to receive the sensor data from each of the one or more sensor devices and wherein the device interface is further couplable to a control interface on each of the one or more effector devices to transmit the control data.

28. A personal medical console for managing a plurality of medical devices,
the plurality of medical devices comprising a plurality of sensor devices and each comprising a console interface couplable to the medical console and configured to communicate with the medical console to provide medical device data to the medical console pertaining to the operation of the medical devices;
the medical console comprising:
a device interface couplable to each of the console interfaces on the plurality of medical devices, the device interface configured to receive the medical device data from each of a plurality of the medical devices;
an analysis engine coupled to the device interface, the analysis engine configured to process the medical device data and determine a secondary physiological characteristic from a combination of the medical device data, convert the secondary physiological characteristic into a data stream; and
a visualisation engine configured to convert the data stream into a visualisation stream for rendering on a display.

29. A personal medical console as claimed in claim 28, wherein the plurality of medical devices further comprise one or more effector devices, wherein the processing the medical device data further comprises determining a divergence of the secondary physiological characteristic to a target health state, and generating control data for output over the device interface for controlling the one or more effector devices responsive to the divergence.

30. A personal medical console as claimed in claim 28, wherein the analysis engine is further configured to merge the medical device data from one or more of the plurality of medical devices into the data stream.

31. A clinical care system comprising the medical console of claim 28 and a plurality of medical devices coupled to the medical console,
the plurality of medical devices comprising sensor devices and/or effector devices and each comprising a console interface configured to communicate with the medical console to provide medical device data to the medical console pertaining to the operation of the medical devices.

32. An interface adapter for interfacing a sensor device to the personal medical console of claim 24,
the sensor device comprising a sensor to sense a physiological characteristic and a data output to output measurement data,
the interface adapter comprising:
a data feed input to receive measurement data from the sensor;
a sensor device interface to output the sensor data, and
wherein the interface adapter is configured to implement a common set of procedures to provide a common data access specification for communicating the sensor data via said sensor device interface.

33. An interface adapter as claimed in claim 32, further comprises a data store configured to store identification data identifying capabilities of a sensor device couplable to the interface adapter.

34. An interface adapter as claimed in claim 33, wherein responsive to a request via the sensor device interface for identification data, the processing engine is configured to read the identification data from the data store and output the identification data to the sensor device interface.

35. A personal medical console for accompanying a patient under medical care and adapted for use by a medical professional, the personal medical console configured to connect to a plurality of medical devices, the medical devices comprising sensor devices and one or more effector devices;
wherein the medical console is configured to:
analyse a patient health state from sensor data received from the plurality of sensor devices;
process the sensor data and generate control data responsive to the processed sensor data for controlling one or more effector devices; and
transmit the control data to the one or more effector devices to control operation of the one or more effector devices.

36. A method of managing a plurality of medical devices using a personal medical console, the medical devices comprising one more sensor devices and one or more effector devices, the method comprising:
monitoring operation of one or more sensor devices coupled to the personal medical console, the one or more sensor devices each comprising a sensor configured to sense physiological characteristics and generate sensor data;
analysing the sensor data received from the plurality of sensor devices by determining a divergence of the sensor data to a target health state; and
outputting control data to one or more effector devices coupled to the medical console responsive to the divergence.

37. A method as claimed in claim 36, wherein the step of analysing further comprises determining a secondary physiological characteristic from a combination of the sensor data, and wherein the step of determining a divergence further comprises determining a divergence of the secondary physiological characteristic to the target health state.

38. A method as claimed in claim 36, further comprising detecting a coupled medical device when coupled to the medical console.

39. A method as claimed in claim 38, further comprising the step of interrogating the coupled medical device to identify capabilities of the medical device.

40. A method as claimed in claim 38, wherein the medical device comprises a sensor device and wherein responsive to detecting the coupled medical device the analysing further comprises analysing the sensor data received from the detected sensor device.

41. A computer program product embodied on a non-transitory computer readable medium carrying computer executable program code configured to implement the method of claim 36.

42. A personal medical console as claimed in claim 24, where the medical console is further configured to generate an alert event responsive to the processed sensor data.
